(19) United States
(12) Patent Application Publication
(10) Pub. No.: US 2008/0119510 A1
(43) Pub. Date: May 22, 2008

(54) Substituted Imidazo [4,5-B] Pyridines As Inhibitors Of Gastric Acid Secretion

(75) Inventors: Christof Brehm, Konstanz (DE); Wilm Buhr, Konstanz (DE); M. Vittoria Chiesa, Konstanz (DE); Andreas Palmer, Singen (DE); Peter Jan Zimmermann, Radolfzell (DE); Wolfgang-Alexander Simon, Konstanz (DE); Stefan Postius, Konstanz (DE); Wolfgang Kromer, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/792,091

(22) PCT Filed: Dec. 6, 2005
(86) PCT No.: PCT/EP05/56509

§ 371 Date: Jul. 6, 2007

(30) Foreign Application Priority Data
Dec. 9, 2004 (EP) 04106442.9

(51) Int. Cl. A61K 31/4745; C07D 471/02

(52) U.S. Cl. 514/303; 546/119

(57) ABSTRACT

The invention provides compounds of the formula (I)

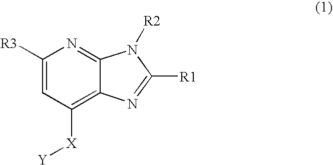

in which the substituents and symbols are as defined in the description. The compounds inhibit the secretion of gastric acid.

### TECHNICAL FIELD

The invention relates to novel compounds which are used in the pharmaceutical industry as active compounds for the production of medicaments.

### BACKGROUND ART

In the International patent application WO 94/22859, heteroannulated imidazole derivatives having a very specific substitution pattern are disclosed, which are said to be suitable as herbizides.

In the German patent DE 1670689, imidazopyridine derivatives are disclosed which have a high activity as herbizides.

The international patent application WO 95/34564 relates to pyridyl imidazole derivatives having an enhanced ability to suppress the activity of angiotensin II.

The syntheses of different imidazo[4,5-b]-pyridines is described inter alia in the Journal of Chemical Society, Perkin Transaction 1, Organic and Bio-organic chemistry, 1992, 21, 2789 or in Receuil des Travaux chimiques des Pays-Bas, 1971, 90(11), 1166, where their effect as bactericides, disinfectants or antiseptics is described.

Purine derived S-adenosyl homocysteine/methylthioadenosine nucleosidase inhibitors with antimicrobial activity are described inter alia in Bioorganic and Medicinal Chemistry Letters, 2004, 14(12), 3165.

In the European Patent EP 0254588 imidazo[4,5-b]-pyridine compounds are described which can be used as drugs for the treatment of gastric and duodenal ulcers.

The International Patent application WO 04/054984 discloses substituted, bicyclic benzimidazole derivatives which compounds are useful for treating gastrointestinal diseases.

### DISCLOSURE OF INVENTION

### Technical Problem

A whole series of compounds are known from the prior art which inhibit gastric acid secretion by blockade of the H+/K+-ATPase. The compounds designated as proton pump inhibitors (PPI's), for example omeprazole, esomeprazole, lansoprazole, pantoprazole or rabeprazole, bind irreversibly to the H+/K+-ATPase. PPI's are available as therapeutics for a long time already. A new class of compounds designated as reversible proton pump inhibitors (rPPI's), as acid pump antagonists (APA's) or as potassium competitive acid blockers (P-CAB's) bind reversibly to the H+/K+-ATPase. Although rPPI's, APA's and P-CAB's are known for more than 20 years and many companies are engaged in their development, no rPPI, APA or P-CAB is at present available for therapy. The technical problem underlying the present invention is therefore to provide potassium competitive acid blockers which can be used in therapy.

### Technical Solution

The invention relates to compounds of the formula 1

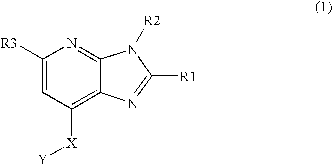

in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl
R2 is hydrogen, 1-4C-alkyl, aryl, 3-7C-cycloalkyl, 3-7C-cycloalkyl-1-4C-alkyl, 1-4C-alkoxycarbonyl, hydroxy-1-4C-alkyl, fluoro-1-4C-alkyl, aryl-1-4C-alkoxy-1-4C-alkyl, hydroxy or 1-4C-alkoxy or mono-or-di-1-4C-alkylamino-carbonyl,
R3 is hydrogen, halogen, fluoro-1-4C-alkyl, carboxyl,—CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, hydroxy-1-4C-alkoxy, 1-4C-alkoxy-1-4C-alkoxy, 1-4C-alkoxy-1-4C-alkoxy-1-4C-alkoxy, fluoro-1-4C-alkoxy, fluoro-1-4C-alkoxy-1-4C-alkoxy, cyano, the group—CO—NR31R32, the group SO₂—NR31R32 or the group Het, where
R31 is hydrogen, hydroxyl, 1-7C-alkyl, hydroxy-1-4C-alkyl, 1-4C-alkoxy-1-4C-alkyl or 3-7C-cycloalkyl, amino and
R32 is hydrogen, 1-7C-alkyl, hydroxy-1-4C-alkyl or 1-4C-alkoxy-1-4C-alkyl, or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol
   where
R33 is hydrogen, 1-4C-alkyl, hydroxy-1-4C-alkyl, 1-4C-alkoxy, 2-4C-alkenyloxy, 1-4C-alkyl-carbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
X is O (oxygen) or NH,
Y has either the meaning —CH₂—Ar
   wherein
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
or Y denotes the group gp

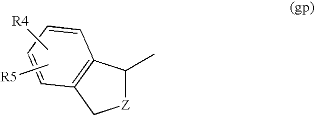

wherein
Z has the meaning —CHR8— or —CHR8—CHR9—
   where in, Ar and/or in the group gp
R4 is hydrogen, halogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, halogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   and wherein
aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
   and the salts of these compounds
   with the proviso that
X does not have the meaning NH, when
R1 is hydrogen or 1-4C-alkyl,
R2 is hydrogen or hydroxy-1-4C-alkyl,
R3 is hydrogen and
Y is unsubstituted —CH₂-phenyl or —CH₂-biphenyl or unsubstituted —CH₂-furyl,
   and
X does not have the meaning O (oxygen), when
R1 is 1-4C-alkyl,
R2 is 1-4C-alkyl,
R3 is hydrogen and
Y is unsubstituted —CH₂-phenyl.

1-4C-Alkyl represents straight-chain or branched alkyl groups having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents groups, which in addition to the oxygen atom contain a straight-chain or branched alkyl group having 1 to 4 carbon atoms. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (—CO-1-4C-alkoxy) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents straight-chain or branched alkenyl groups having 2 to 4 carbon atoms. Examples which may be mentioned are the 2-butenyl, 3-butenyl, 1-propenyl and the 2-propenyl group (allyl group).

2-4C-Alkynyl represents straight-chain or branched alkynyl groups having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-4C-alkyl groups, which is substituted by one or more fluorine atoms. Examples which may be mentioned are the trifluoromethyl, the difluoromethyl, and the 2,2,2-trifluoroethyl group.

Hydroxy-1-4C-alkyl represents aforementioned 1-4C-alkyl groups, which are substituted by a hydroxy group. Examples which may be mentioned are the hydroxymethyl, the 2-hydroxyethyl and the 3-hydroxypropyl group.

1-4C-Alkylcarbonyl represents a group, which in addition to the carbonyl group contains one of the aforementioned 1-4C-alkyl groups. An example which may be mentioned is the acetyl group.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

Aryl-1-4C-alkoxy denotes an aryl-substituted 1-4C-alkoxy radical. An example which may be mentioned is the benzyloxy radical.

Aryl-1-4C-alkoxy-1-4C-alkyl denotes one of the aforementioned 1-4C-alkyl groups, which is substituted by one of the aforementioned aryl-1-4C-alkoxy radicals. An example which may be mentioned is the benzyloxymethyl radical.

Mono- or di-1-4C-alkylamino-carbonyl represents a carbonyl group, to which one of the aforementioned mono- or di-1-4C-alkylamino groups is bonded. Examples which may be mentioned are the dimethylaminocarbonyl, the diethylaminocarbonyl and the diisopropylaminocarbonyl radicals.

Halogen within the meaning of the invention is bromo, chloro and fluoro.

1-4C-Alkoxy-1-4C-alkoxy represents one of the aforementioned 1-4C-alkoxy groups, which is substituted by a further 1-4C-alkoxy group. Examples which may be mentioned are the groups 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. Examples which may be mentioned are the group 2-(methoxy)ethoxymethyl (CH₃—O—CH₂—CH₂—O—CH₂—) and the 2-(ethoxy)ethoxymethyl (CH₃—CH₂—O—CH₂—CH₂—O—CH₂—) radicals.

1-4C-Alkoxy-1-4C-alkoxy-1-4C-alkoxy represents one of the aforementioned 1-4C-alkoxy-1-4C-alkoxy groups which is substituted by one of the aforementioned 1-4C-alkoxy groups. Examples which may be mentioned are the group 2-(methoxy)ethoxymethoxy (CH₃—O—CH₂—CH₂—O—CH₂—O—) and the 2-(ethoxy)ethoxymethoxy (CH₃—CH₂—O—CH₂—CH₂—O—CH₂—O—) radicals.

Fluoro-1-4C-alkoxy represents one of the aforementioned 1-4C-alkoxy groups, which is substituted by one or more fluorine atoms. Examples of fluoro-1-4C-alkoxy groups which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy, the fluoromethoxy and preferably the difluoromethoxy group.

Fluoro-1-4C-alkoxy-1-4C-alkyl represents one of the aforementioned 1-4C-alkyl groups, which is substituted by a fluoro-1-4C-alkoxy group. Examples which may be mentioned are the 1,1,2,2-tetrafluoroethoxymethyl, the 2,2,2-trifluoromethoxymethyl, the trifluoromethoxyethyl and the difluoromethoxymethyl group.

1-7C-Alkyl represents straight-chain or branched alkyl groups having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl and the methyl group.

2-4C-Alkenyloxy represents groups, which in addition to the oxygen atom contain one of the abovementioned 2-4C-alkenyl groups. Examples, which may be mentioned, are the 2-butenyloxy, 3-butenyloxy, 1-propenyloxy and the 2-propenyloxy group (allyloxy group).

Carboxy-1-4C-alkyl represents 1-4C-alkyl groups which are substituted by a carboxyl group. Examples, which may be mentioned, are the carboxymethyl and the 2-carboxyethyl group.

1-4C-Alkoxycarbonyl-1-4C-alkyl represents 1-4C-alkyl groups, which are substituted by one of the abovementioned 1-4C-alkoxycarbonyl groups. Examples, which may be mentioned, are the Methoxycarbonylmethyl and the ethoxycarbonylmethyl group.

Aryl-1-4C-alkyl denotes an aryl-substituted 1-4C-alkyl radical. An example which may be mentioned is the benzyl radical.

1-4C-Alkylcarbonylamino represents an amino group to which a 1-4C-alkylcarbonyl group is bonded. Examples which may be mentioned are the propionylamino (C₃H₇C(O)NH—) and the acetylamino group (acetamido group) (CH₃C(O)NH—).

1-4C-Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-(methoxy)ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-4C-alkoxy-1-4C-alkoxycarbonyl groups. Examples which may be mentioned are the 2-(methoxy)ethoxycarbonylamino and the 2-(ethoxy)ethoxycarbonylamino group.

2-7C-Alkenyl represents straight-chain or branched alkenyl groups having 2 to 7 carbon atoms. Examples which may be mentioned are the 2-butenyl, 3-butenyl, 1-propenyl, the 2-propenyl (allyl) and the vinyl group. The aforementioned 2-4C-alkenyl groups are preferred.

2-7C-Alkenyl represents straight-chain or branched alkenyl groups having 2 to 7 carbon atoms. Examples which may be mentioned are the 2-butenyl, 3-butenyl, 1-propenyl, the 2-propenyl (allyl) and the vinyl group. The aforementioned 2-4C-alkenyl groups are preferred.

Oxo-substituted 1-4C-alkoxy represents a 1-4C-alkoxy group, which instead of a methylene group contains a carbonyl group. An example which may be mentioned is the 2-oxopropoxy group.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkyl-1-4C-alkoxy represents one of the aforementioned 1-4C-alkoxy groups, which is substituted by one of the aforementioned 3-7C-cycloalkyl groups. Examples which may be mentioned are the cyclopropylmethoxy, the cyclobutylmethoxy and the cyclohexylethoxy group.

Hydroxy-1-4C-alkoxy represents aforementioned 1-4C-alkoxy groups, which are substituted by a hydroxy group. A preferred example which may be mentioned is the 2-hydroxyethoxy group.

1-4C-Alkoxy-1-4C-alkoxy-1-4C-alkoxy represents one of the aforementioned 1-4C-alkoxy groups, which is substituted by one of the aforementioned 1-4C-alkoxy-1-4C-alkoxy groups. A preferred example which may be mentioned is the methoxyethoxyethoxy group.

3-7C-Cycloalkoxy-1-4C-alkoxy represents one of the aforementioned 1-4C-alkoxy groups, which is substituted by one of the aforementioned 3-7C-cycloalkoxy groups. Examples which may be mentioned are the cyclopropoxymethoxy, the cyclobutoxymethoxy and the cyclohexyloxyethoxy group.

3-7C-Cycloalkyl-1-4C-alkoxy-1-4C-alkoxy represents one of the aforementioned 1-4C-alkoxy groups, which is substituted by one of the aforementioned 3-7C-cycloalkyl-1-4C-alkoxy groups. Examples which may be mentioned are the cyclopropylmethoxyethoxy, the cyclobutylmethoxyethoxy and the cyclohexylethoxyethoxy group.

1-4C-Alkylcarbonyloxy represents a 1-4C-alkylcarbonyl group which is bonded to an oxygen atom. An example which may be mentioned is the acetoxy group (CH₃CO—O—).

1-4C-Alkylcarbonyloxy-1-4C-alkyl represents one of the aforementioned 1-4C-alkyl groups, which is substituted by one of the aforementioned 1-4C-alkylcarbonyloxy groups. An example which may be mentioned is the acetoxymethyl group (CH₃CO—O—CH₂).

Halo-1-4C-alkoxy represents 1-4C-alkoxy groups which are completely or mainly substituted by halogen. “Mainly” in this connection means that more than half of the hydrogen atoms in the 1-4C-alkoxy groups are replaced by halogen atoms. Halo-1-4C-alkoxy groups are primarily chloro- and/or in particular fluoro-substituted 1-4C-alkoxy groups. Examples of halogen-substituted 1-4C-alkoxy groups which may be mentioned are the 2,2,2-trichloroethoxy, the hexachloroisopropoxy, the pentachloroisopropoxy, the 1,1,1-trichloro-3,3,3-trifluoro-2-propoxy, the 1,1,1-trichloro-2-methyl-2-propoxy, the 1,1,1-trichloro-2-propoxy, the 3-bromo-1,1,1-trifluoro-2-propoxy, the 3-bromo-1,1,1-trifluoro-2-butoxy, the 4-bromo-3,3,4,4-tetrafluoro-1-butoxy, the chlorodifluoromethoxy, the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group.

Mono- or di-1-4C-alkylamino-1-4C-alkylcarbonyloxy represents a 1-4C-alkylcarbonyloxy group, which is substituted by one of the aforementioned mono- or di-1-4C-alkylamino groups. Examples, which may be mentioned, are the dimethylamino-methylcarbonyloxy and the dimethylamino-ethylcarbonyloxy group.

1-4C-Alkoxy-1-4C-alkylcarbonyloxy represents one of the aforementioned 1-4C-alkylcarbonyloxy radicals which is substituted by one of the aforementioned 1-4C-alkoxy groups. An example, which may be mentioned, is the methoxymethylcarbonyloxy group.

Hydroxy-1-4C-alkoxy represents one of the aforementioned 1-4C-alkoxy radicals which is substituted by a hydroxy group. Examples which may be mentioned are the the 2-hydroxyethoxy and the 3-hydroxypropoxy group.

Fluoro-1-4C-alkoxy has the meaning as defined for fluoro-1-4C-alkoxy-1-4C-alkyl. A preferred example which may be mentioned is the difluoromethoxy group.

Fluoro-1-4C-alkoxy-1-4C-alkoxy represents one of the aforementioned 1-4C-alkoxy groups, which is substituted by one of the aforementioned fluoro-1-4C-alkoxy groups. Examples of fluoro-1-4C-alkoxy-1-4C-alkoxy radicals which may be mentioned are the difluoromethoxyethoxy or the 1,1,1-trifluoroethoxyethoxy group.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

One embodiment (embodiment a) of the invention comprises compounds of the formula 1, in which
X is O (oxygen),
R1, R2, R3 and Y have the meanings as indicated in the outset, and the salts of these compounds.

Another embodiment (embodiment b) of the invention comprises compounds of the formula 1, in which
X is NH,
R1, R2, R3 and Y have the meanings as indicated in the outset, and the salts of these compounds.

Another embodiment (embodiment c) of the invention comprises compounds of the formula 1, in which
R3 is hydrogen,
R1, R2, X and Y have the meanings as indicated in the outset, and the salts of these compounds.

Another embodiment (embodiment d) of the invention comprises compounds of the formula 1, in which
R3 is halogen, fluoro-1-4C-alkyl, carboxyl,—CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, hydroxy-1-4C-alkoxy, 1-4C-alkoxy-1-4C-alkoxy, 1-4C-alkoxy-1-4C-alkoxy-1-4C-alkoxy, fluoro-1-4C-alkoxy, fluoro-1-4C-alkoxy-1-4C-alkoxy, cyano, the group—CO—NR31 R32, the group SO₂—NR31R32 or the group Het, where
R31 is hydrogen, hydroxyl, 1-7C-alkyl, hydroxy-1-4C-alkyl, 1-4C-alkoxy-1-4C-alkyl or 3-7C-cycloalkyl, amino and
R32 is hydrogen, 1-7C-alkyl, hydroxy-1-4C-alkyl or 1-4C-alkoxy-1-4C-alkyl,
   or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol
   where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
R1, R2, X and Y have the meanings as indicated in the outset, and the salts of these compounds.

Another embodiment (embodiment e) of the invention comprises compounds of the formula 1, in which
Y is unsubstituted —CH₂-phenyl or —CH₂-biphenyl or unsubstituted —CH₂-furyl, and R1, R2, R3 and X have the meanings as indicated in the outset, and the salts of these compounds.

Another embodiment (embodiment f) of the invention comprises compounds of the formula 1, in which
R1, R2, R3, X and Y have the meanings as indicated in the outset,
   with the proviso that
Y is unsubstituted —CH₂-phenyl or —CH₂-biphenyl or unsubstituted —CH₂-furyl, and the salts of these compounds.

Preferred are those compounds of the formula 1, in which
R1 is 1-4C-alkyl, 3-7C-cycloalkyl, hydroxy-1-4C-alkyl or 1-4C-alkoxy-1-4C-alkyl
R2 is hydrogen,1-4C-alkyl, hydroxy or 1-4C-alkoxy,
R3 is hydrogen, halogen, carboxyl,—CO-1-4C-alkoxy, hydroxy-1-4C-alkyl, 1-4C-alkoxy-1-4C-alkyl, cyano, 1-4C-alkoxy, hydroxy-1-4C-alkoxy, 1-4C-alkoxy-1-4C-alkoxy, the group —CO—NR31R32, the group SO₂—NR31R32 or the group Het,
   where
R31 is hydrogen, 1-7C-alkyl, hydroxy-1-4C-alkyl, 1-4C-alkoxy-1-4C-alkyl, 3-7C-cycloalkyl or
amino and
R32 is hydrogen or 1-7C-alkyl,
   or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol and dihydroimidazol,
   where
R33 is hydrogen or 1-4C-alkyl,
R34 is hydrogen or 1-4C-alkyl
R35 is hydrogen or 1-4C-alkyl
X is O (oxygen) or NH,
Y has either the meaning —CH₂—Ar—
   wherein
Ar is phenyl substituted by R4 and R5
   or
Y denotes the group gp

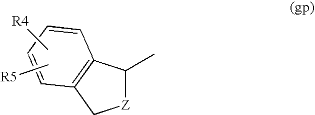

wherein
Z has the meaning —CHR8—

### Where in Ar and/or in the group gp

R4 is hydrogen, halogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, halogen, 1-4C-alkyl or 1-4C-alkoxy
R8 is hydrogen, hydroxyl, 1-4C-alkoxy, hydroxy-1-4C-alkoxy, 1-4C-alkoxy-1-4C-alkoxy, amino, mono-or di-1-4C-alkylamino,
   and the salts of these compounds
   with the proviso that
X does not have the meaning NH, when
R1 is 1-4C-alkyl,
R2 is hydrogen,
R3 is hydrogen and
Y is unsubstituted —CH₂-phenyl,
   and
X does not have the meaning O (oxygen), when
R1 is 1-4C-alkyl,
R2 is 1-4C-alkyl,
R3 is hydrogen and
Y is unsubstituted —CH₂-phenyl.

Particularly preferred are those compounds of the formula 1, in which
R1 is 1-4C-alkyl
R2 is 1-4C-alkyl
R3 is hydrogen, halogen, carboxyl,—CO-1-4C-alkoxy, 1-4C-alkoxy, 1-4C-alkoxy-1-4C-alkoxy or the group—CO—NR31R32,
   where
R31 is hydrogen, 1-4C-alkyl or hydroxy-1-4C-alkyl and
R32 is hydrogen or 1-4C-alkyl,
X is NH,
Y has the meaning —CH₂—Ar—
   wherein
Ar is phenyl substituted by R4 and R5
   wherein
R4 is 1-4C-alkyl
R5 is 1-4C-alkyl
   and the salts of these compounds.

In one aspect (aspect a) the invention relates to compounds of the formula 1a

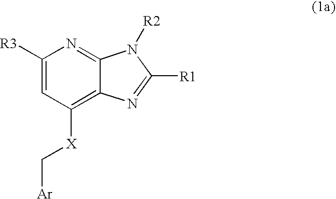

in which
R1, R2, R3, X and Ar have the meanings as indicated in the outset and the salts of these compounds.

Among the compounds of the formula 1a, preferred compounds are those of the formula 1a-1

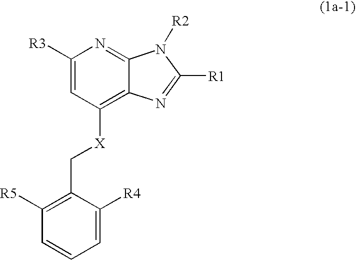

in which
R1, R2, R3, R4, R5 and X have the meanings as indicated in the outset and the salts of these compounds.

Among the compounds of the formula 1, particularly preferred are those compounds of the formula 1a-1,

in which
R1 is 1-4C-alkyl, 3-7C-cycloalkyl, hydroxy-1-4C-alkyl or 1-4C-alkoxy-1-4C-alkyl
R2 is hydrogen,1-4C-alkyl, hydroxy or 1-4C-alkoxy,
R3 hydrogen, halogen, carboxyl,—CO-1-4C-alkoxy, hydroxy-1-4C-alkyl, 1-4C-alkoxy-1-4C-alkyl, cyano, 1-4C-alkoxy, hydroxy-1-4C-alkoxy, 1-4C-alkoxy-1-4C-alkoxy, the group—CO—NR31R32, the group SO₂—NR31R32 or the group Het,
   where
R31 is hydrogen, 1-7C-alkyl, hydroxy-1-4C-alkyl, 1-4C-alkoxy-1-4C-alkyl, 3-7C-cycloalkyl or
amino and
R32 is hydrogen or 1-7C-alkyl,
   or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol and dihydroimidazol,
   where
R33 is hydrogen or 1-4C-alkyl,
R34 is hydrogen or 1-4C-alkyl
R35 is hydrogen or 1-4C-alkyl
R4 is hydrogen, halogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, halogen, 1-4C-alkyl or 1-4C-alkoxy
X is O (oxygen) or NH,
   and the salts of these compounds
   with the proviso that
R4 does not have meaning of hydrogen, when
R1 is 1-4C-alkyl,
R2 is hydrogen,
R3 is hydrogen,
R5 is hydrogen and
X is NH
   and
R4 does not have meaning of hydrogen, when
R1 is 1-4C-alkyl,
R2 is 1-4C-alkyl,
R3 is hydrogen,
R5 is hydrogen and
X is O (oxygen).

Compounds of the formula 1, which are to be emphasized, are those compounds of the formula 1a-1, in which
R1 is 1-4C-alkyl
R2 is 1-4C-alkyl
R3 hydrogen, halogen, carboxyl,—CO-1-4C-alkoxy, 1-4C-alkoxy, 1-4C-alkoxy-1-4C-alkoxy or the group—CO—NR31R32,
   where
R31 is hydrogen, 1-4C-alkyl or hydroxy-1-4C-alkyl and
R32 is hydrogen or 1-4C-alkyl,
R4 is 1-4C-alkyl
R5 is 1-4C-alkyl
X is NH,
   and the salts of these compounds.

In another aspect (aspect b) the invention relates to compounds of the formula 1b

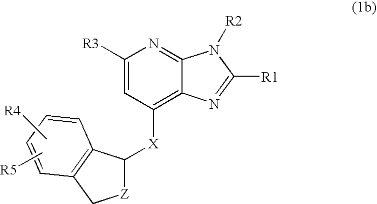

in which
R1, R2, R3, R4, R5, X and Z have the meanings as indicated in the outset and the salts of these compounds.

The compounds of the formula 1b have up to three centers of chirality in the parent structure. The invention thus provides all feasible enantiomers in any mixing ratio, including the pure enantiomers, which are a preferred subject matter of the invention.

Among the compounds of the formula 1b, preferred compounds are those of the formula 1 b-1

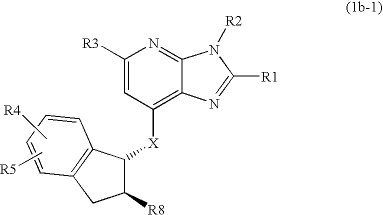

in which
R1, R2, R3, R4, R5, R8 and X have the meanings as indicated in the outset, and the salts of these compounds.

Among the compounds of the formula 1, particularly preferred are those compounds of the formula 1 b-1, in which
R1 is 1-4C-alkyl, 3-7C-cycloalkyl, hydroxy-1-4C-alkyl or 1-4C-alkoxy-1-4C-alkyl
R2 is hydrogen,1-4C-alkyl, hydroxy or 1-4C-alkoxy,
R3 hydrogen, halogen, carboxyl,—CO-1-4C-alkoxy, hydroxy-1-4C-alkyl, 1-4C-alkoxy-1-4C-alkyl, cyano, 1-4C-alkoxy, hydroxy-1-4C-alkoxy, 1-4C-alkoxy-1-4C-alkoxy, the group—CO-NR31R32, the group SO₂-NR31R32 or the group Het,
   where
R31 is hydrogen, 1-7C-alkyl, hydroxy-1-4C-alkyl, 1-4C-alkoxy-1-4C-alkyl or 3-7C-cycloalkyl and
R32 is hydrogen or 1-7C-alkyl,
   or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy
R8 is hydrogen, hydroxyl, 1-4C-alkoxy, hydroxy-1-4C-alkoxy, 1-4C-alkoxy-1-4C-alkoxy, amino, mono-or di-1-4C-alkylamino,
X is O (oxygen) or NH,
   and the salts of these compounds.

Compounds of the formula 1, which are to be emphasized, are those compounds of the formula 1 b-1, in which
R1 is 1-4C-alkyl
R2 is 1-4C-alkyl
R3 hydrogen, halogen, carboxyl,—CO-1-4C-alkoxy, 1-4C-alkoxy, 1-4C-alkoxy-1-4C-alkoxy or the group—CO—NR31R32,
   where
R31 is hydrogen, 1-4C-alkyl or hydroxy-1-4C-alkyl and
R32 is hydrogen or 1-4C-alkyl,
R4 is hydrogen, fluoro, or 1-4C-alkyl
R5 is hydrogen, fluoro, or 1-4C-alkyl
R8 is hydroxy or 1-4C-alkoxy,
X is O (oxygen) or NH,
   and the salts of these compounds.

The compounds according to the invention can be synthesized from corresponding starting compounds, for example according to the reaction schemes given below. The synthesis is carried out in a manner known to the expert, for example as described in more detail in the following examples.

The compounds of the general formula 1a with X═NH can be obtained for example according to the reaction sequence as shown in scheme 1.

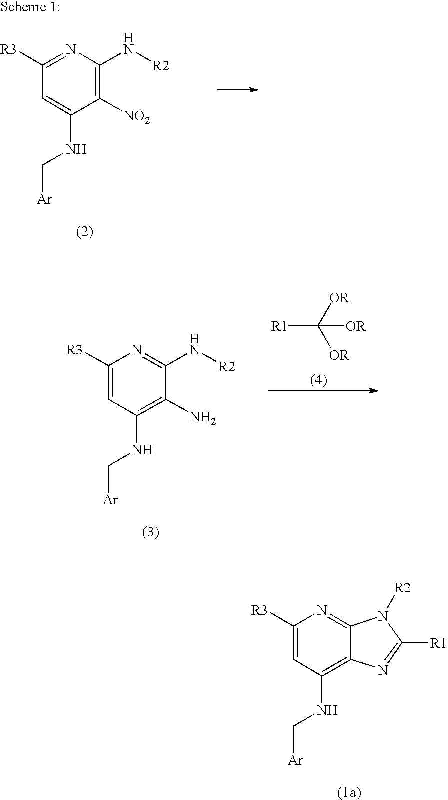

Compounds of the formula 2 can be reduced under conditions known to the expert, for example using hydrogen over Raney nickel, to compounds of the formula 3. The final cyclization step to compounds of the formula 1 is performed under acidic conditions, for example using ortho-esters of the formula 4, wherein R1 is for example hydrogen or a 1-4C-alkyl radical and the substituents R are for example 1-4C-alkyl radicals.

The compounds of the general formula 1b with X═NH can be obtained for example by reacting substituted imidazo[4,5-b]pyridines of the general formula 5 with epoxyindanes or 1a,2,3,7b-tetrahydronaphtho[1,2-b]oxirenes. In scheme 2, the reaction mode is exemplified for epoxyindanes of the formula 6 carrying any desired substituent R4 and R5, which leads to the preferred compounds of the formula 1 b-1. Epoxyindan is described for example in W. F. Whitmore; A. I. Gebhart, J. Am. Chem. Soc. 1942, 64, 912. In general, substituted alkyl-, alkoxy- or halogeno-epoxyindanes can be prepared from the corresponding substituted indenes by methods known from literature (e.g. epoxidation).

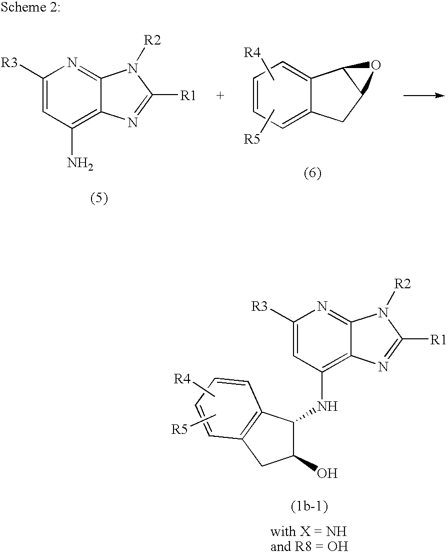

Starting compounds of the formula 2 can be prepared for example as shown in scheme 3 by reacting compounds of the formula 7, wherein Lg is a suitable leaving group, like a halogen atom, for example a chlorine atom, with compounds of the formula 8, wherein L is a suitable leaving group like for example a suitable halogen atom, like for example a chlorine atom. The leaving group Lg in the resulting compounds of the formula 9 can be substituted by an amino functionality by reaction with an amine carrying a suitable substituent R2 to form compounds of the formula 2.

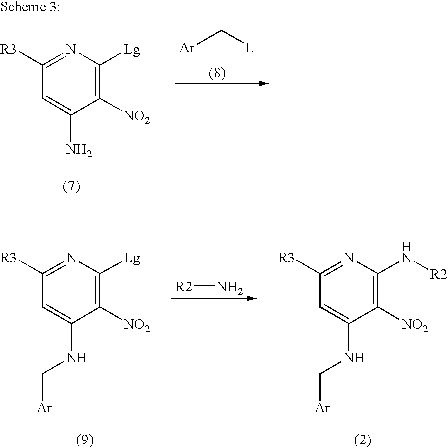

Starting compounds of the formula 5 can be prepared using methodologies known to a person skilled in the art which can achieve the removal of the group Ar—CH₂— (for example an benzyl group) in compounds of the formula 1a, for example by palladium-catalyzed hydrogenation, as shown in scheme 4.

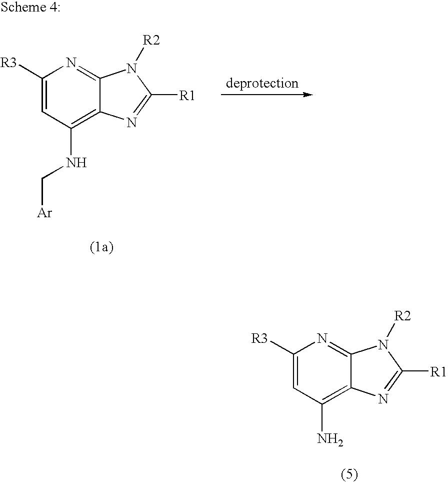

Starting compounds of the formula 7 are known, for example from R. J. Rousseau, R. K. Robins, J. Het. Chem. 1965, 2, 196-201 or can be prepared in an analogous manner.

The compounds of the general formula 1a with X═O (oxygen) can be obtained for example according to the reaction sequence as shown in scheme 5.

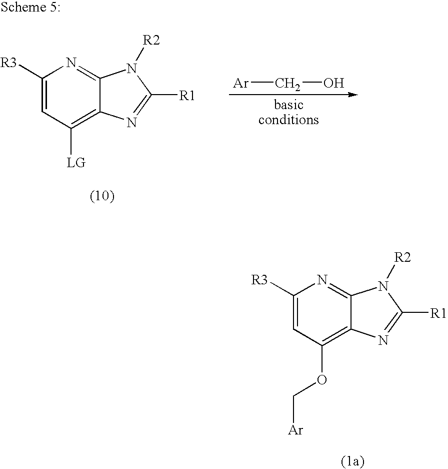

In compounds of the formula 10 the leaving group LG (for example a nitro group or a halogen group) can be substituted by alcohols of the formula Ar—CH₂—OH (for example benzyl alcohol) under basic conditions.

The compounds of the general formula 1b with X═O (oxygen) can be synthesized by reaction of imidazo[4,5-b]pyridines of the general formula 10 with 1-hydroxy-indanes or 1-hydroxy-1,2,3,4-tetrahydronaphthalines. In scheme 6, the reaction mode is exemplified for a 2-hydroxy-protected indane-1,2-diol of the formula 11 carrying any desired substituent R4 and R5, which leads after deprotection to the preferred compounds of the formula 1 b-2. Suitable protected indane-1,2-diols (as protecting group can serve for example an acetyl group) of the formula 11 are described for example by S. Sengupta, S. Mondal, in Tetrahedron Letters 40, 1999, 3469-3470.

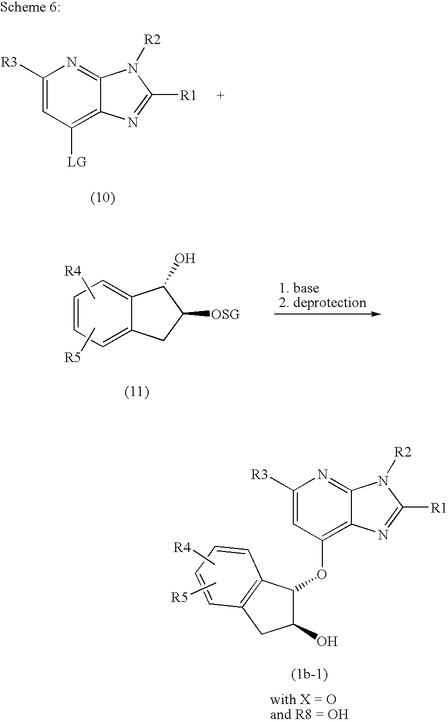

Starting compounds of the formula 10 are known, for example from G. Cristalli, M. Grifantini, Nucleosides & Nucleotides 4, 1985, 625-639 or can be prepared in an analogous manner.

The compounds of the formula 1 b-1 with R8=OH as shown in scheme 2 and scheme 6 can be further be derivatized by methods known to the expert to other compounds of the formula 1 b-1 with R8 having the other meanings as indicated in the outset.

The reaction steps outlined above are carried out in a manner known per se, e. g. as described in more detail in the examples. The derivatization, if any, of the compounds obtained according to the above Scheme 1, 2, 3, 4, 5 and 6 (e.g. conversion of a group R3 into another group R3 or conversion of a hydroxyl group into an alkoxy or ester group) is likewise carried out in a manner known per se. If for example compounds of the formula 1a or 1b where R3=—CO-1-4C-alkoxy or R3=—CO—NR31R32 are desired, an appropriate derivatization can be performed in a manner known per se (e. g. metal catalysed carbonylation of the corresponding halogen, for example chloro, compound or conversion of an ester or a carboxylic acid into an amide) for example at the stage of compounds of the formula 2, 3, 5 or 10 or preferably at the stage of compounds of the formula 1a or 1b respectively (scheme 1 and scheme 2). If for example compounds of the formula 1a are desired where R3=hydroxy-1-4C-alkoxy, 1-4C-alkoxy-1-4C-alkoxy, 1-4C-alkoxy-1-4C-alkoxy-1-4C-alkoxy or fluoro-1-4C-alkoxy-1-4C-alkoxy an appropriate derivatization can be performed in a manner known per se, for example by nucleophilic substitution of R3 at the stage of a compound of the formula 1a, 3 or preferably at the stage of a compound of the formula 2, wherein R3 is a suitable leaving group, like for example a chloro atom.

### Advantageous Effects

The excellent gastric protective action and the gastric acid secretion-inhibiting action of the compounds according to the invention can be demonstrated in investigations on animal experimental models. The compounds of the formula 1 according to the invention investigated in the model mentioned below have been provided with numbers which correspond to the numbers of these compounds in the examples.

### Testing of the Secretion-inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 1 | 3.0 | >30 |
| 2 | 3.0 | >30 |
| 3 | 3.0 | >30 |
| 5 | 3.0 | >30 |
| 6 | 3.0 | >30 |

### Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01 N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

### MODE(S) FOR CARRYING OUT THE INVENTION

The examples below serve to illustrate the invention in more detail without limiting it. Further compounds of the formula 1 whose preparation is not described explicitly can likewise be prepared in an analogous manner or in a manner known per se to the person skilled in the art, using customary process techniques. The compounds named expressly as examples, and the salts of these compounds, are preferred subject matter of the invention. The abbreviation min stands for minute(s), h stands for hour(s), m.p. stands for melting point and ee for enantiomeric excess.

### I. Final Compounds of the Formula 1

### 1. 7-(2-Ethyl-6-methylbenzyl)amino-2,3-dimethyl-3H-imidazo[4,5-b]pyridine

A solution of 5-chloro-7-(2-ethyl-6-methylbenzyl)amino-2,3-dimethyl-3H-imidazo[4,5-b]pyridine (500 mg, 1.50 mmol) and triethylamine (210 μl, 1.50 mmol) in methanol (50 ml) was hydrogenated over 10% Pd/C (50 mg) (1 bar H₂) for 18 h at 50° C. The catalyst was filtered off and the filtrate concentrated in vacuum. The residue was purified by column chromatography on silica gel using toluene:dioxane (3:1, v/v) and crystallized from petroleum ether to afford 370 mg (84%) of the title compound as a white solid. m.p. 105-109° C.

### 2. 5-(Dimethylaminocarbonyl)-7-(2-ethyl-6-methylbenzyl)amino-2,3-dimethyl-3H-imidazo[4,5-b]pyridine

O-(1H-Benzotriazol-1-yl)-N,N,N′,N′-tetramethyluronium tetrafluoroborate (TBTU) (337 mg, 1.05 mmol) was added to a suspension of 7-(2-ethyl-6-methylbenzyl)amino-2,3-dimethyl-3H-imidazo[4,5-b]pyridine-5-carboxylic acid (240 mg, 0.70 mmol) in dichloromethane (18 ml) and the mixture was stirred at room temperature for 12 h.

Dichloromethane (10 ml) and dimethylamine (5 M in tetrahydrofuran) (560 μl, 2.8 mmol) were added and the resulting clear solution was stirred for further 5 h at room temperature. The solution was poured onto water (50 ml) and extracted with dichloromethane (2×10 ml). The collected organic layers were washed with a solution of sodium hydroxide (2 M) (2×5 ml), dried over magnesium sulfate, and concentrated in vacuum. The residue was crystallized from petroleum ether to afford 220 mg (86%) of the title compound as a white solid. m.p. 133-136° C.

### 3. 5-(Aminocarbonyl)-7-(2-ethyl-6-methylbenzyl)amino-2,3-dimethyl-3H-imidazo[4,5-b]pyridine

O-(1H-Benzotriazol-1-yl)-N,N,N′,N′-tetramethyluronium tetrafluoroborate (TBTU) (482 mg, 1.50 mmol) was added to a suspension of 7-(2-ethyl-6-methylbenzyl)amino-2,3-dimethyl-3H-imidazo[4,5-b]pyridine-5-carboxylic acid (340 mg, 1.00 mmol) in dichloromethane (30 ml) and the mixture was stirred at room temperature for 12 h.

An aqueous ammonium chloride solution (25%) (616 μl, 4.00 mmol) was added and the reaction mixture was stirred for further 2 h at room temperature. The solution was poured onto ice-cooled water (70 ml) and extracted with dichloromethane (2×20 ml). The collected organic layers were washed with a solution of sodium hydroxide (2 M) (10 ml) and water (10 ml), dried over magnesium sulfate, and concentrated in vacuum. The residue was crystallized from diisopropyl ether to afford 300 mg (89%) of the title compound as a white solid. m.p. 237-239° C.

### 4. 7-(2-Ethyl-6-methylbenzyl)amino-2,3-dimethyl-5-(methylaminocarbonyl)-3H-imidazo[4,5-b]pyridine

O-(1H-Benzotriazol-1-yl)-N,N,N′,N′-tetramethyluronium tetrafluoroborate (TBTU) (482 mg, 1.50 mmol) was added to a suspension of 7-(2-ethyl-6-methylbenzyl)amino-2,3-dimethyl-3H-imidazo[4,5-b]pyridine-5-carboxylic acid (340 mg, 1.00 mmol) in dichloromethane (30 ml) and the mixture was stirred at room temperature for 12 h.

Methyl amine (33% in methanol) (748 μl, 6.00 mmol) was added and the reaction mixture was stirred for further 6 h at room temperature. The solution was poured onto ice-cooled water (70 ml) and extracted with dichloromethane (2×20 ml). The collected organic layers were washed with a solution of sodium hydroxide (2 M) (10 ml) and water (10 ml), dried over magnesium sulfate, and concentrated in vacuum. The residue was crystallized from diisopropyl ether to afford 290 mg (83%) of the title compound as a white solid. m.p. 236-238° C.

### 5. 7-(2-Ethyl-6-methylbenzyl)amino-5-(2-hydroxyethylaminocarbonyl)-2,3-dimethyl-3H-imidazo[4,5-b]pyridine

O-(1H-Benzotriazol-1-yl)-N,N,N′,N′-tetramethyluronium tetrafluoroborate (TBTU) (482 mg, 1.50 mmol) was added to a suspension of 7-(2-ethyl-6-methylbenzyl)amino-2,3-dimethyl-3H-imidazo[4,5-b]pyridine-5-carboxylic acid (340 mg, 1.00 mmol) in dichloromethane (30 ml) and the mixture was stirred at room temperature for 12 h.

Ethanolamine (245 μl, 4.00 mmol) was added and the reaction mixture was stirred for further 6 h at room temperature. The solution was poured onto ice-cooled water (70 ml) and extracted with dichloromethane (2×20 ml). The collected organic layers were washed with a solution of sodium hydroxide (2 M) (10 ml) and water (10 ml), dried over magnesium sulfate, and concentrated in vacuum. The residue was crystallized from diisopropyl ether to afford 350 mg (92%) of the title compound as a white solid. m.p. 256-258° C.

### 6. 7-(2-Ethyl-6-methylbenzyl)amino-5-methoxy-2,3-dimethyl-3H-imidazo[4,5-b]pyridine

A solution of 4-(2-ethyl-6-methylbenzyl)amino-6-methoxy-2-methylamino-3-nitro-pyridine (0.99 g, 3.00 mmol) in methanol (80 ml) and dimethylformamide (20 ml) was hydrogenated over raney nickel (aqueous solution) (1.50 g) (1 bar H₂) for 20 h at 40° C. The catalyst was filtered off and the filtrate concentrated in vacuum. The residue was filtered through a pad of silica gel using toluene:dioxane (3:1, v/v) to afford 0.62 g (69%) of a dark green solid. The product (3-amino-4-(2-ethyl-6-methylbenzyl)amino-6-methoxy-2-methylamino-pyridine) was used as such for the following reaction step without further purification.

Hydrochloric acid (6 M) (100 μl) and trimethyl orthoacetate (283 μl, 2.20 mmol) were added to a solution of 3-amino-4-(2-ethyl-6-methylbenzyl)amino-6-methoxy-2-methylamino-pyridine (330 mg, 1.10 mmol) in ethanol (7.5 ml). The mixture was heated to reflux for 1 h, further trimethyl orthoacetate (283 μl, 2.20 mmol) was then added and the reaction mixture was heated to reflux for an additional hour. After cooling the mixture was stirred for 12 h at room temperature. The reaction mixture was poured onto ice-cooled water (100 ml) and extracted with dichloromethane (4×20 ml). The collected organic layers were washed with water (10 ml), dried over magnesium sulfate, and concentrated in vacuum. The residue was purified by column chromatography on silica gel using toluene:dioxane (5:1, v/v) and crystallized from petroleum ether to afford 135 mg (38%) of the title compound as a pale green solid. m.p. 165-166° C.

### 7. 7-(2-Ethyl-6-methylbenzyl)amino-5-methoxyethoxy-2,3-dimethyl-3H-imidazo[4,5-b]pyridine Fumarate

A suspension of 4-(2-ethyl-6-methylbenzyl)amino-6-methoxyethoxy-2-methylamino-3-nitro-pyridine (0.30 g, 0.80 mmol) in methanol (50 ml) was hydrogenated over raney nickel (aqueous solution) (250 mg) (1 bar H₂) for 20 h at 40° C. The catalyst was filtered off and the filtrate concentrated in vacuum to afford a green solid (3-amino-4-(2-ethyl-6-methylbenzyl)amino-6-methoxyethoxy-2-methylamino-pyridine), which was used as such in the following reaction step without further purification.

Hydrochloric acid (6 M) (150 μl) and trimethyl orthoacetate (206 μl, 1.60 mmol) were added to a solution of 3-amino-4-(2-ethyl-6-methylbenzyl)amino-6-methoxyethoxy-2-methylamino-pyridine (250 mg, 0.80 mmol) in ethanol (5 ml) and the mixture was heated to reflux for 1 h. Further trimethyl orthoacetate (283 μl, 2.20 mmol) was added, the reaction mixture was heated to reflux for an additional hour and this procedure was repeated once again. The reaction mixture was poured onto ice-cooled water (100 ml) and extracted with dichloromethane (4×20 ml). The collected organic layers were washed with water (10 ml), dried over magnesium sulfate, and concentrated in vacuum. The residue was purified by column chromatography on silica gel using toluene:dioxane (10:1, v/v) yielding a green oil which was dissolved in acetone (3 ml) and treated with a solution of fumaric acid (32 mg, 0.27 mmol) in acetone (3 ml). The solvent was removed and the residue was crystallized from petroleum ether to afford 80 mg (21%) of the title compound as a pale brown solid. m.p. 161-162° C.

### 8. 5-Chloro-7-(2-ethyl-6-methylbenzyl)amino-2,3-dimethyl-3H-imidazo[4,5-b]pyridine

A suspension of 6-chloro-4-(2-ethyl-6-methylbenzyl)amino-2-methylamino-3-nitro-pyridine (4.02 g, 12.0 mmol) in ethanol (650 ml) and dimethylformamide (50 ml) was hydrogenated over raney nickel (aqueous solution) (2.20 g) (1 bar H₂) for 20 h at 40° C. The catalyst was filtered off and the filtrate was concentrated in vacuum to afford 3.60 g of a brown solid. The product (3-amino-6-chloro-4-(2-ethyl-6-methylbenzyl)amino-2-methylamino-pyridine) was used as such in the next reaction step without further purification.

Concentrated hydrochloric acid (500 μl) and trimethyl orthoacetate (2.32 ml, 18.0 mmol) were added to a solution of 3-amino-6-chloro-4-(2-ethyl-6-methylbenzyl)amino-2-methylamino-pyridine (3.60 g, 12.0 mmol) in ethanol (100 ml). The mixture was heated to reflux for 45 min at which point further trimethyl orthoacetate (2.32 ml, 18.0 mmol) was added. After a period of 45 min, a last batch of trimethyl orthoacetate (1.15 ml, 9.00 mmol) was added, the reaction mixture was heated to reflux for additional 30 min, and stirred at room temperature for 12 h. The reaction mixture was concentrated in vacuum, the residue was poured onto ice-cooled water (200 ml), and extracted with dichloromethane (4×50 ml). The collected organic layers were washed with water (25 ml), dried over magnesium sulfate, and concentrated in vacuum. The residue was purified by column chromatography on silica gel using toluene:dioxane (15:1, v/v) and crystallized from petroleum ether to afford 2.43 g (62%) of the title compound as a pale brown solid. m.p. 157-159° C.

### 9. Ethyl-7-(2-ethyl-6-methylbenzyl)amino-2,3-dimethyl-3H-imidazo[4,5-b]pyridine-5-carboxylic Ester

To a solution of 5-chloro-7-(2-ethyl-6-methylbenzyl)amino-2,3-dimethyl-3H-imidazo[4,5-b]pyridine (0.50 g, 1.50 mmol) in ethanol (40 ml) and N,N-dimethylformamide (10 ml) were added palladium(II) acetate (17 mg, 0.08 mmol) and 1,3-bis-(diphenylphophino)propane (35 mg, 0.008 mmol). The mixture was transferred to an autoclave and carbonylated (20 bar carbon monoxide pressure, 140° C.) for 15 h. The reaction mixture was cooled down, filtered over a pad of silicagel and concentrated to a tenth of the original volume. The residue was poured onto an ice-cooled solution of ammonium chloride (200 ml, 10%) resulting a brown precipitate. Purification of this precipitate by column chromatography on silica gel using toluene:dioxane (10:1, v/v) yielded 0.40 g (73%) of the title compound as a colourless solid. m.p. 153-156° C.

10. 7-(2-ethyl-6-methylbenzyl)amino-2,3-dimethyl-3H-imidazo[4,5-b]pyridine-5-carboxylic Acid

Ethyl-7-(2-ethyl-6-methylbenzyl)amino-2,3-dimethyl-3H-imidazo[4,5-b]pyridine-5-carboxylic ester (0.30 g, 0.80 mmol) was dissolved in dioxan and an aqueous solution of lithium hydroxide (0.20 ml, 6 N) was added. The reaction mixture was heated at 85° C. for 4 h, after cooling down poored onto water (150 ml), neutralized by adding hydrochloric acid (2N), and extracted with dichloromethane (4×50 ml). The collected organic layers were dried over magnesium sulfate, and concentrated in vacuum. The resulting white solid was crystallized from petroleum ether to afford 0.26 g (96%) of the title compound as a pale brown solid. m.p. 235-238° C.

### II. Starting Compounds

### A. 2,6-Dichloro-4-(2-ethyl-6-methylbenzyl)amino-3-nitro-pyridine

Solution 1: A solution of 4-amino-2,6-dichloro-3-nitro-pyridine (8.38 g, 40.0 mmol) in tetrahydrofuran (65 ml) was added to a suspension of sodium hydride (1.92 g, 48.0 mmol) in tetrahydrofuran (35 ml) and stirred for 2 h at room temperature.

Solution 2: Sodium iodide (6.66 g, 44.0 mmol) was added to a solution of 2-ethyl-6-methylbenzyl chloride (7.42 g, 44.0 mmol) in tetrahydrofuran (35 ml). The reaction mixture was stirred for 2 h at room temperature.

Solution 2 was then added to the solution 1 under ice cooling. Stirring was continued for 30 min, tetrahydrofuran was partially removed under vacuum, and the residue was poured onto water (800 ml). Extraction of the aqueous phase with ethyl acetate (4×200 ml), washing of the collected organic phases with water (50 ml), drying over magnesium sulfate, and distillation of the solvent in vacuum delivered an oil as the crude product. Further purification by column chromatography on silica gel using toluene:petroleum ether (10:1, v/v) afforded 9.30 g (68%) of the title compound as a yellow solid. m.p. 118-121° C.

### B. 6-Chloro-4-(2-ethyl-6-methylbenzyl)amino-2-methylamino-3-nitro-pyridine

A solution of methylamine in ethanol (33%) (4.50 ml, 36.0 mmol) and triethylamine (4.50 ml) were added to a suspension of 2,6-dichloro-4-(2-ethyl-6-methylbenzyl)amino-3-nitro-pyridine (5.10 g, 15.0 mmol) in 2-methoxyethanol (90 ml). The reaction mixture was heated to 55° C. for 1 h forming a clear solution.

The solution was concentrated in vacuum, diluted with a small amount of ethanol, and poured onto ice (600 ml). An orange precipitate was formed which was filtered off, washed with water and dried at 40° C. in vacuum for 12 h. 4.93 g (98%) of the title compound were obtained as an orange solid. m.p. 152-157° C.

### C. 4-(2-Ethyl-6-methylbenzyl)amino-6-methoxy-2-methylamino-3-nitro-pyridine

A solution of sodium methylate in methanol (30%) (0.69 ml, 3.60 mmol) was added to the suspension of 6-chloro-4-(2-ethyl-6-methylbenzyl)amino-2-methylamino-3-nitro-pyridine (0.34 g, 1.00 mmol) in methanol (15 ml). The reaction mixture was stirred for 3 h at room temperature and poured onto ice-cooled water (150 ml). A yellow precipitate was formed. The aqueous mixture was neutralized using aqueous hydrochloric acid and the precipitate was filtered off, washed with water, and dried at 40° C. in vacuum to afford 0.30 g (91%) of the title compound as a yellow solid. m.p. 147-149° C.

### D. 4-(2-Ethyl-6-methylbenzyl)amino-6-methoxyethoxy-2-methylamino-3-nitro-pyridine

Sodium hydride (145 mg, 3.63 mmol) was carefully added to 2-methoxyethanol (10 ml). The resulting solution was stirred for 90 min and 6-chloro-4-(2-ethyl-6-methylbenzyl)amino-2-methylamino-3-nitro-pyridine (0.34 g, 1.00 mmol) was added. The reaction mixture was heated to reflux for 90 min and poured onto ice-cooled water (100 ml). The aqueous mixture was extracted with ethyl acetate (4×25 ml), the collected organic phases were washed with water (20 ml), dried over magnesium sulfate, and concentrated in vacuum. The residue was purified by column chromatography on silica gel using toluene:dioxane (20:1, v/v) to afford 0.32 g (86%) of the title compound as a yellow solid. m.p. 93-95° C.

### INDUSTRIAL APPLICABILITY

The compounds of the formula 1, 1a, 1a-1, 1b and 1b-1 and their salts (active compounds according to the invention) have valuable pharmacological properties which make them commercially utilizable. In particular, they exhibit marked inhibition of gastric acid secretion and an excellent gastric and intestinal protective action in warm-blooded animals, in particular humans. In this connection, the active compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the active compounds according to the invention surprisingly prove to be clearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the above-mentioned diseases.

The invention furthermore includes the use of the active compounds according to the invention for the treatment and/or prophylaxis of the abovementioned diseases.

A further subject of the invention are medicaments which comprise one or more compounds of the active compounds according to the invention.

The medicaments are prepared by processes which are known per se and familiar to the person skilled in the art. As medicaments, the pharmacologically active compounds according to the invention are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds according to the invention can be administered orally, parenterally or percutaneously.

In general, it has proven advantageous in human medicine to administer the active compound according to the invention in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the active compound according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the active compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable anti-bacterial co-components which may be mentioned are, for example, mezlocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the active compounds according to the invention are suited for a free or fixed combination with those medicaments (e.g. certain antiinflammatories and antirheumatics, such as NSAIDs), which are known to have a certain ulcerogenic potency. In addition, the active compounds according to the invention are suited for a free or fixed combination with motility-modifying drugs.

1. A compound of the formula 1 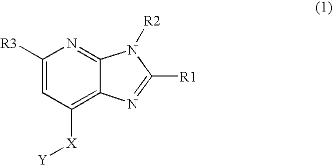
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, hydroxy-1-4C-alkyl, fluoro-1-4C-alkyl, aryl-1-4C-alkoxy-1-4C-alkyl, hydroxy or 1-4C-alkoxy or mono-or-di-1-4C-alkylamino-carbonyl,
R3 is hydrogen, halogen, fluoro-1-4C-alkyl, carboxyl,—CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, hydroxy-1-4C-alkoxy, 1-4C-alkoxy-1-4C-alkoxy, 1-4C-alkoxy-1-4C-alkoxy-1-4C-alkoxy, fluoro-1-4C-alkoxy, fluoro-1-4C-alkoxy-1-4C-alkoxy, cyano, the group—CO—NR31R32, the group SO₂—NR31R32 or the group Het,
where
R31 is hydrogen, hydroxyl, 1-7C-alkyl, hydroxy-1-4C-alkyl, 1-4C-alkoxy-1-4C-alkyl or 3-7C-cycloalkyl, amino and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino, or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
X is O (oxygen) or NH,
Y has either the meaning —CH₂—Ar,
wherein
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
or Y denotes the group gp 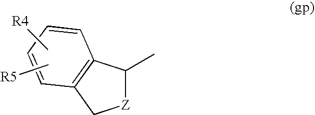
wherein
Z has the meaning —CHR8— or —CHR8—CHR9—
where, in Ar and/or in the group gp,
R4 is hydrogen, halogen, 1-4C-alkyl, hydroxy-1-4C-alkyl, 1-4C-alkoxy, 2-4C-alkenyloxy, 1-4C-alkyl-carbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, halogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
or a salt thereof
with the proviso that
X does not have the meaning NH, when
R1 is hydrogen or 1-4C-alkyl,
R2 is hydrogen or hydroxy-1-4C-alkyl,
R3 is hydrogen and
Y is unsubstituted —CH₂-phenyl or —CH₂-biphenyl or unsubstituted —CH₂-furyl,
and
X does not have the meaning O (oxygen), when
R1 is 1-4C-alkyl,
R2 is 1-4C-alkyl,
R3 is hydrogen and
Y is unsubstituted —CH₂-phenyl.

2. A compound of the formula 1 as claimed in claim 1, in which
R1 is 1-4C-alkyl, 3-7C-cycloalkyl, hydroxy-1-4C-alkyl or 1-4C-alkoxy-1-4C-alkyl,
R2 is hydrogen, 1-4C-alkyl, hydroxy or 1-4C-alkoxy,
R3 is hydrogen, halogen, carboxyl,—CO-1-4C-alkoxy, hydroxy-1-4C-alkyl, 1-4C-alkoxy-1-4C-alkyl, cyano, 1-4C-alkoxy, hydroxy-1-4C-alkoxy, 1-4C-alkoxy-1-4C-alkoxy, the group—CO—NR31R32, the group SO2-NR31R32 or the group Het,
where
R31 is hydrogen, 1-7C-alkyl, hydroxy-1-4C-alkyl, 1-4C-alkoxy-1-4C-alkyl, 3-7C-cycloalkyl or amino and
R32 is hydrogen or 1-7C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol and dihydroimidazol,
where
R33 is hydrogen or 1-4C-alkyl,
R34 is hydrogen or 1-4C-alkyl
R35 is hydrogen or 1-4C-alkyl
X is O (oxygen) or NH,
Y has either the meaning —CH₂—Ar—,
wherein
Ar is phenyl substituted by R4 and R5
or
Y denotes the group gp 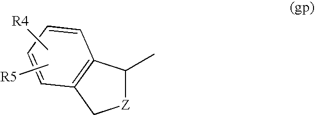
wherein
Z has the meaning —CHR8—
where, in Ar and/or in the group gp,
R4 is hydrogen, halogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, halogen, 1-4C-alkyl or 1-4C-alkoxy,
R8 is hydrogen, hydroxyl, 1-4C-alkoxy, hydroxy-1-4C-alkoxy, 1-4C-alkoxy-1-4C-alkoxy, amino, mono-or di-1-4C-alkylamino,
or a salt thereof
with the proviso that
X does not have the meaning NH, when
R1 is 1-4C-alkyl,
R2 is hydrogen,
R3 is hydrogen and
Y is unsubstituted —CH₂-phenyl,
and
X does not have the meaning O (oxygen), when
R1 is 1-4C-alkyl,
R2 is 1-4C-alkyl,
R3 is hydrogen and
Y is unsubstituted —CH₂-phenyl.

3. A compound of the formula 1 as claimed in claim 1, in which
R1 is 1-4C-alkyl,
R2 is 1-4C-alkyl,
R3 is hydrogen, halogen, carboxyl,—CO-1-4C-alkoxy, 1-4C-alkoxy, 1-4C-alkoxy-1-4C-alkoxy or the group —CO—NR31R32,
where
R31 is hydrogen, 1-4C-alkyl or hydroxy-1-4C-alkyl and
R32 is hydrogen or 1-4C-alkyl,
X is NH,
Y has the meaning —CH₂—Ar—,
wherein
Ar is phenyl substituted by R4 and R5
wherein
R4 is 1-4C-alkyl,
R5 is 1-4C-alkyl,
or a salt thereof.

4. A compound of the formula 1 as claimed in claim 1, characterized by the formula 1a-1 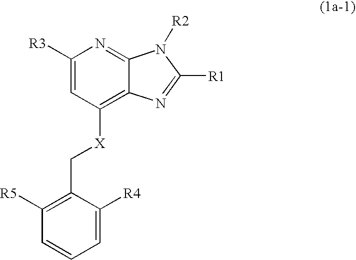
in which
R1, R2, R3, R4, R5 and X have the meanings as indicated in claim 1,
or a salt thereof.

5. A compound of the formula 1 as claimed in claim 1, characterized by the formula 1a-1 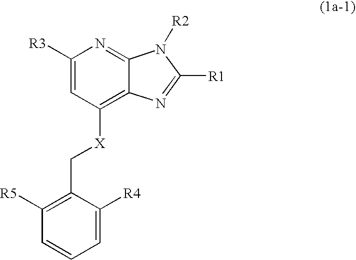
in which
R1 is 1-4C-alkyl, 3-7C-cycloalkyl, hydroxy-1-4C-alkyl or 1-4C-alkoxy-1-4C-alkyl,
R2 is hydrogen, 1-4C-alkyl, hydroxy or 1-4C-alkoxy,
R3 hydrogen, halogen, carboxyl,—CO-1-4C-alkoxy, hydroxy-1-4C-alkyl, 1-4C-alkoxy-1-4C-alkyl, cyano, 1-4C-alkoxy, hydroxy-1-4C-alkoxy, 1-4C-alkoxy-1-4C-alkoxy, the group—CO—NR31R32, the group SO2-NR31R32 or the group Het,
where
R31 is hydrogen, 1-7C-alkyl, hydroxy-1-4C-alkyl, 1-4C-alkoxy-1-4C-alkyl, 3-7C-cycloalkyl or amino and
R32 is hydrogen or 1-7C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol and dihydroimidazol,
where
R33 is hydrogen or 1-4C-alkyl,
R34 is hydrogen or 1-4C-alkyl,
R35 is hydrogen or 1-4C-alkyl,
R4 is hydrogen, halogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, halogen, 1-4C-alkyl or 1-4C-alkoxy,
X is O (oxygen) or NH,
or a salt thereof,
with the proviso that
R4 does not have meaning of hydrogen, when
R1 is 1-4C-alkyl,
R2 is hydrogen,
R3 is hydrogen,
R5 is hydrogen and
X is NH,
and
R4 does not have meaning of hydrogen, when
R1 is 1-4C-alkyl,
R2 is 1-4C-alkyl,
R3 is hydrogen,
R5 is hydrogen and
X is O (oxygen).

6. A compound of the formula 1 as claimed in claim 1, characterized by the formula 1a-1, 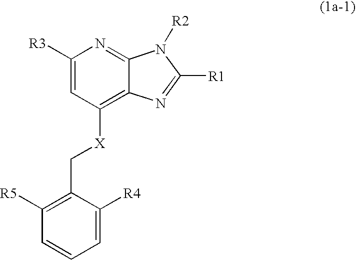
in which
R1 is 1-4C-alkyl,
R2 is 1-4C-alkyl,
R3 hydrogen, halogen, carboxyl,—CO-1-4C-alkoxy, 1-4C-alkoxy, 1-4C-alkoxy-1-4C-alkoxy or the group —CO-NR31R32,
where
R31 is hydrogen, 1-4C-alkyl or hydroxy-1-4C-alkyl and
R32 is hydrogen or 1-4C-alkyl,
R4 is 1-4C-alkyl,
R5 is 1-4C-alkyl,
X is NH,
or a salt thereof.

7. A compound of the formula 1 as claimed in claim 1, characterized by the formula 1b, 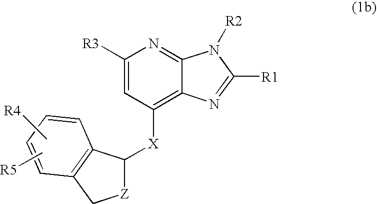
in which R1, R2, R3, R4, R5, X and Z have the meanings as indicated in claim 1,
or a salt thereof.

8. A compound of the formula 1 as claimed in claim 1, characterized by the formula 1b-1, 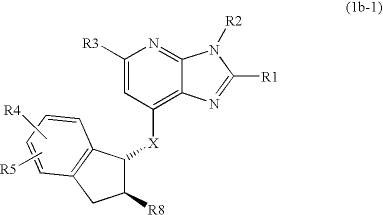
in which
R1, R2, R3, R4, R5, R8 and X have the meanings as indicated in claim 1,
or a salt thereof.

9. A pharmaceutical composition comprising a compound as claimed in claim 1 and/or a pharmacologically acceptable salt thereof together with a pharmaceutically acceptable auxiliary and/or excipient.

10. (canceled)

11. A method of preventing or treating a gastrointestinal disorder in a patient comprising administering to a patient in need thereof a compound of formula I as claimed in claim 1 or a pharmacologically acceptable salt thereof.
